(19) United States
(12) Patent Application Publication
(10) Pub. No.: US 2026/0061224 A1
(43) Pub. Date: Mar. 5, 2026

(54) Improvements in or Relating to Medical Device Apparatus

(71) Applicant: XStrahl Limited, Walsall (GB)

(72) Inventors: Emily Louise McNee, Armitage (GB); Shaun Melvyn Phillips, Peterborough (GB); Andrew James Cowie, Glen Parva (GB); Bruce James Renfrew, Leicester (GB)

(73) Assignee: XStrahl Limited, Walsall (GB)

(21) Appl. No.: 19/120,856

(22) PCT Filed: Oct. 10, 2023
(86) PCT No.: PCT/GB2023/052630

§ 371 Date: Apr. 14, 2025

(30) Foreign Application Priority Data
Oct. 14, 2022 (GB) 2215243.3

(51) Int. Cl. A61N 5/10

(52) U.S. Cl. CPC A61N 5/1083

(57) ABSTRACT

A support structure for a medical device system includes a first arm portion with a structure attachment portion; and a second arm portion that supports a medical device. The first arm portion is pivotably attachable to the structure attachment portion. The second arm portion is pivotably attachable to the first arm portion. The first arm portion includes first and second biasing devices to bias the first arm portion and second arm portion. The support structure may have a mounting apparatus for a medical device attachable to the second arm portion. A medical device system, a support structure for a device system, a device support system and associated methods are also provided.

The present invention relates to a support structure for a medical device system, a medical device system, a support structure for a device system, and a method of operating the same.

X-ray systems for delivering X-ray radiotherapy for superficial therapy and treatment of skin conditions are known. Such X-ray systems include base units upon which X-ray head support arms are mounted. The base unit is typically provided on wheels or castors, which allows the X-ray system to be easily transported. The X-ray head support arm is also arranged to pivot and rotate relative to the base unit, such that the X-ray head can be positioned to be as close as possible to the patient.

The X-ray head support arms include a number of components for the operation of the X-ray head, such as power cables, communication cables etc., as well as componentry that allows the arm to pivot and rotate to different positions.

While such X-ray systems are capable of delivering X-ray radiotherapy to patients, they are known to be bulky and difficult to balance and hold in an equilibrium position. The increased size and weight of the components of the support arms means that larger, more expensive, components, such as brakes and support struts, are required to balance the arm. Such known systems are thus expensive to manufacture.

The present inventors have appreciated the shortcomings in the above-described X-ray systems.

According to a first aspect of the present invention there is provided a support structure for a medical device system, comprising:
a first arm portion, the first arm portion including a structure attachment portion; and
a second arm portion, the second arm portion being configured to support a medical device,
wherein the first arm portion is pivotably attachable to the structure attachment portion, and may pivot between a first position and a second position, and
wherein the second arm portion is pivotably attachable to the first arm portion, and may pivot between a first position and a second position, and
wherein the first arm portion includes:
   a first biasing device, the first biasing device being configured to bias the first arm portion towards its first position; and
   a second biasing device, the second biasing device being configured to bias the second arm portion towards its first position.

The support structure may be a support arm.

The support structure may be a moveable support structure. The support structure may be configurable to assume two or more positions. The support structure may be configurable to assume a plurality of positions.

The support structure may be for an X-ray medical device system. The support structure may be for a radiotherapy X-ray medical device system.

The support structure may be configured to support a mounting apparatus. The support structure may be a mounting apparatus support structure. The support structure may be a mounting apparatus support structure for a medical device system. The support structure may be a mounting apparatus support structure for a medical device.

The support structure may be configured to support a medical device. The second arm portion may be configured to support a medical device. The second arm portion may be configured to support a mounting apparatus for a medical device.

The medical device may be an X-ray tube. The medical device may be an X-ray tube head. The medical device may be a radiotherapy X-ray tube. The medical device may be a radiotherapy X-ray tube head. The medical device may be an end-window X-ray tube. The medical device may be an end-window X-ray tube head. The medical device may be an end-window radiotherapy X-ray tube. The medical device may be an end-window radiotherapy X-ray tube head.

The X-ray tube head may include an applicator. The applicator contacts the skin of a patient in use to ensure that a predetermined distance exists between the X-ray tube window and the patient's skin. The applicator also exerts a pressure on the patient's skin to flatten the area to be exposed to the X-rays.

The first arm portion may be an elongate structure. The first arm portion may be an elongate member.

The second arm portion may be an elongate structure. The second arm portion may be an elongate member.

The first arm portion may include a housing. The housing may be a body portion. The body portion may be a housing. The housing may be an elongate member. The body portion may be an elongate member.

The second arm portion may include a housing. The housing may be a body portion. The body portion may be a housing. The housing may be an elongate member. The body portion may be an elongate member.

The first arm portion and the second arm portion may be pivotably attachable to one another. The first arm portion and the second arm portion may be pivotably connectable to one another.

The first arm portion may have a longitudinal axis. The longitudinal axis extending between a first, lower, end, and a second, upper, end. The first arm portion may have a lateral axis. The lateral axis extending between a first, left, side, and a second, right, side.

The second arm portion may have a longitudinal axis. The longitudinal axis extending between a first, lower, end, and a second, upper, end. The second arm portion may have a lateral axis. The lateral axis extending between a first, left, side, and a second, right, side.

The structure attachment portion may be configured to allow the support structure to be attachable to a structure. The structure may be a component of a medical device system. The structure may be a base unit for a medical device system. The structure may be component of a building, such as a wall, or the like. The structure may be component of a vehicle, such as the wall of a building, such as a wall, or the like.

The first arm portion may be attachable to the structure attachment portion.

The structure attachment portion may be located at the first, lower, end of the first arm portion.

The structure attachment portion may be fixedly attachable to the structure. The structure attachment portion may be fixedly attachable to the structure by one or more fastening devices. The one or more fastening devices may be mechanical fastening devices, or the like.

The structure attachment portion may include a through passage, channel, or conduit. The through passage, channel, or conduit, may allow cabling, or the like, to pass through the structure attachment portion.

The second arm portion may be configured to provide support to a mounting apparatus. The second arm portion may include a mounting apparatus. The mounting apparatus may be a mounting apparatus for a medical device.

The second arm portion may include a mounting apparatus attachment portion. The second arm portion may be attachable to the mounting apparatus attachment portion.

The mounting apparatus attachment portion may be located at the second, upper, end of the second arm portion.

The mounting apparatus attachment portion may include a through passage, channel, or conduit. The through passage, channel, or conduit, may allow cabling, or the like, to pass through the mounting apparatus attachment portion.

The first arm portion may be pivotably attachable to the structure attachment portion by a first pivot device. The first arm portion may include a pivot device. The structure attachment portion may include a pivot device.

The first pivot device may be located at the first, lower, end of the first arm portion.

The first pivot device may be configured to allow the first arm portion to pivot with respect to the structure attachment portion. The first pivot device may be configured to allow the first arm portion to pivot with respect to the structure attachment portion between the first position and the second position.

The first pivot device may include a first member and a second member. The first member may be attachable to the structure attachment portion. The second member may be attachable to the first member. The second member may be pivotably attachable to the first member. The second member may be attachable to the first arm portion. The second member may be attachable to the housing of the first arm portion.

The first member may be a pivot support member, or support member. The second member may be a pivot member.

The first pivot device may be a hinge, hinge member, or hinge device.

The first pivot device may include a through passage, channel, or conduit. The through passage, channel, or conduit, may allow cabling, or the like, to pass through the first pivot device. The first member of the first pivot device may include a through passage, channel, or conduit. The through passage, channel, or conduit, may allow cabling, or the like, to pass through the first pivot device.

The first pivot device may be lockable. The first pivot device may be lockable, such that the first and second pivot members cannot move relative to one another. The first and second members of the first pivot device may be configured such that they may be locked in position relative to one another.

The first pivot device may be lockable at more than one position. That is, the first pivot device may be lockable with the first and second pivot members of the first pivot device at different positions relative to one another.

The first pivot device may include a locking system. The locking system may be operable to lock the first and second pivot members relative to one another.

The first member of the first pivot device may include a locking aperture. The locking aperture may be arranged to allow a fastening member to pass therethrough. The first member of the first pivot device may include two locking apertures. The first member of the first pivot device may a first locking aperture and a second locking aperture. The first locking aperture and the second locking aperture may be angularly offset from one another. In this arrangement the first pivot device may be lockable with the first and second pivot members of the first pivot device at different positions relative to one another.

The locking apertures may be arranged to allow a fastening member to pass therethrough.

The second member of the first pivot device may include a locking aperture. The aperture may be arranged to receive and engage with a fastening member. The fastening member may be a bolt, or the like.

The first and second members of the first pivot device may be arranged such that their respective locking apertures may be aligned, and a fastening member may be inserted into the apertures to lock the members together. This position may be a locked position.

The first and second members of the first pivot device may be arranged such that locking aperture of the second member may be aligned with the first or second locking aperture of the first member, and a fastening member may be inserted into one of the apertures to lock the members together. This position may be a locked position.

The first pivot device may be arranged to allow the first arm portion to move between its first position and its second position.

The first pivot device may be configured such that the first and second pivot members may move between a first position and a second position.

The first member of the first pivot device may include a pivot aperture. The pivot aperture may be an elongate aperture. The pivot aperture may be a partial arc. The pivot aperture may be arc-shaped. The pivot aperture may be an elongated arc. The pivot aperture may be arc-shaped slot. The pivot aperture may be arranged to allow a fastening member to pass therethrough.

The second member of the first pivot device may include a pivot aperture. The pivot aperture may be arranged to receive and engage with a fastening member. The pivot aperture of the second member may be a circular aperture. The fastening member may be a bolt, or the like.

The first and second members of the first pivot device may be arranged such that their respective pivot apertures may be aligned, and a fastening member may be inserted into the apertures. In this arrangement the fastening member is fixed to the second member of the first pivot device, and free to move relative to the first member of the first pivot device. The fastening member may be configured to abut either end of the pivot aperture of the first member. A first abutment between the fastening member and the first member defines the first position of the pivot device, and a second abutment between the fastening member and the first member defines the second position of the pivot device. The first and second abutments are at opposite ends of the pivot aperture of the first member. In this arrangement the length of the arc defines the range of pivot, or angular, motion of the first and second members of the first pivot device. The fastening member may be a bolt, or the like.

The first pivot device may be arranged in a second position. The second position may be an operating position. In this position the first biasing device applies a bias to the first arm portion, and the first arm portion may move between its first position and its second position. In this position the first arm portion is biased by the first biasing device.

The first pivot device may be locked in the operating position. The first pivot device may be locked in the operating position by aligning the locking aperture of the second member with second locking aperture of the first member, and using a fastening member to lock the members together. The locked operating position may be a transit position. The transit position allows the components of the support arm to be transported safely.

The second arm portion may be pivotably attachable to the first arm portion by a second pivot device. The second arm portion may include a pivot device. The first arm portion may include the first pivot device and a second pivot device.

The second pivot device may be located at the first, lower, end of the second arm portion.

The second pivot device may be configured to allow the second arm portion to pivot with respect to the first arm portion. The second pivot device may be configured to allow the second arm portion to pivot with respect to the first arm portion between the first position and the second position.

The second pivot device may include a first member and a second member. The first member may be attachable to the first arm portion. The second member may be attachable to the first member. The second member may be pivotably attachable to the first member. The second member may be attachable to the second arm portion. The second member may be attachable to the housing of the second arm portion.

The first member may be a pivot support member, or support member. The second member may be a pivot member.

The second pivot device may be a hinge, hinge member, or hinge device.

The second pivot device may include a through passage, channel, or conduit. The through passage, channel, or conduit, may allow cabling, or the like, to pass through the second pivot device. The second member of the second pivot device may include a through passage, channel, or conduit. The through passage, channel, or conduit, may allow cabling, or the like, to pass through the first pivot device.

The second pivot device may be lockable. The second pivot device may be lockable, such that the first and second pivot members cannot move relative to one another. The first and second members of the second pivot device may be configured such that they may be locked in position relative to one another.

The second pivot device may be lockable at more than one position. That is, the second pivot device may be lockable with the first and second pivot members of the second pivot device at different positions relative to one another.

The second pivot device may include a locking system. The locking system may be operable to lock the first and second pivot members relative to one another.

The second member of the second pivot device may include a locking aperture. The locking aperture may be arranged to allow a fastening member to pass therethrough. The second member of the second pivot device may include two locking apertures. The second member of the second pivot device may a first locking aperture and a second locking aperture. The first locking aperture and the second locking aperture may be angularly offset from one another. In this arrangement the second pivot device may be lockable with the first and second pivot members of the second pivot device at different positions relative to one another.

The locking apertures may be arranged to allow a fastening member to pass therethrough.

The first member of the second pivot device may include a locking aperture. The aperture may be arranged to receive and engage with a fastening member. The fastening member may be a bolt, or the like.

The first and second members of the second pivot device may be arranged such that their respective locking apertures may be aligned, and a fastening member may be inserted into the apertures to lock the members together. This position may be a locked position.

The first and second members of the second pivot device may be arranged such that locking aperture of the first member may be aligned with the first or second locking aperture of the second member, and a fastening member may be inserted into one of the apertures to lock the members together. This position may be a locked position.

The second pivot device may be arranged to allow the second arm portion to move between its first position and its second position.

The second pivot device may be configured such that the first and second pivot members may move between a first position and a second position.

The second member of the second pivot device may include a pivot aperture. The pivot aperture may be an elongate aperture. The pivot aperture may be a partial arc. The pivot aperture may be arc-shaped. The pivot aperture may be an elongated arc. The pivot aperture may be arranged to allow a fastening member to pass therethrough.

The first member of the second pivot device may include a pivot aperture. The pivot aperture may be arranged to receive and engage with a fastening member. The pivot aperture of the first member may be a circular aperture. The fastening member may be a bolt, or the like.

The first and second members of the second pivot device may be arranged such that their respective pivot apertures may be aligned, and a fastening member may be inserted into the apertures. In this arrangement the fastening member is fixed to the first member of the second pivot device, and free to move relative to the second member of the second pivot device. The fastening member may be configured to abut either end of the pivot aperture of the second member. A first abutment between the fastening member and the second member defines the first position of the pivot device, and a second abutment between the fastening member and the second member defines the second position of the pivot device. The first and second abutments are at opposite ends of the pivot aperture of the second member. In this arrangement the length of the arc defines the range of pivot, or angular, motion of the first and second members of the second pivot device. The fastening member may be a bolt, or the like.

The second pivot device may be arranged in a second position. The second position may be an operating position. In this position the second biasing device applies a bias to the second arm portion, and the second arm portion may move between its first position and its second position. In this position the second arm portion is biased by the second biasing device.

The second pivot device may be locked in the operating position. The second pivot device may be locked in the operating position by aligning the locking aperture of the first member with second locking aperture of the second member, and using a fastening member to lock the members together. The locked operating position may be a transit position. The transit position allows the components of the support arm to be transported safely.

The first and second pivot devices may be locked in a first position. The first position may be an assembly position. The assembly position allows the components of the support arm to be assembled. In this position the first and second biasing devices do not apply any bias to the first arm portion or the second arm portion. In this position the first arm portion and the second arm portion are substantially parallel to one another. In this position the first arm portion and the second arm portion are substantially colinear with one another.

The first and second pivot devices may be locked in a second position. The second position may be a transit position. The transit position allows the support arm to be transported safely. In this position the first and second biasing devices apply a bias to the first arm portion and the second arm portion. In this position the first arm portion and the second arm portion are pivoted to their second positions.

The first pivot device may be arranged to allow the first arm portion to pivot through between 30 and 70 degrees of rotation. The first pivot device may be arranged to allow the first arm portion to pivot through 50 degrees of rotation.

The second pivot device may be arranged to allow the second arm portion to pivot through between 100 and 120 degrees of rotation. The second pivot device may be arranged to allow the second arm portion to pivot through 110 degrees of rotation.

The first and second pivot devices may be attachable to one another. The second member of the first pivot device may be attachable to the first member of the second pivot device. The first and second pivot devices may be secured to one another. The second member of the first pivot device may be secured to the first member of the second pivot device.

The first and second pivot devices may be attachable to one another by a connecting member. The first and second pivot devices may be secured to one another by a connecting member. The connecting member may be a first connecting member.

The connecting member may be fastened to each of the first and second pivot devices. The connecting member may be attachable to each of the first and second pivot devices by one or more fastening devices. The connecting member may be secured to each of the first and second pivot devices by one or more fastening devices. The one or more fastening devices may be mechanical fastening devices, or the like.

The connecting member may include a threaded portion. The connecting member may include a threaded portion at each end thereof. The connecting member may be fastened to the first and second pivot devices by one or more nut members. The one or more nut members being configured to engage with the threaded portion of the connecting member.

The one or more nut members being configured to engage with the threaded portion of the connecting member and at least a portion of each pivot device.

The connecting member may be an elongate member. The connecting member may be an elongate rod. The connecting member may be a rod member. The connecting member may be cylindrical elongate member. The connecting member may be a solid metal member.

The second pivot device and the mounting apparatus attachment portion may be attachable to one another. The second member of the second pivot device and the mounting apparatus attachment portion may be attachable to one another. The second pivot device and the mounting apparatus attachment portion may be secured to one another. The second member of the second pivot device and the mounting apparatus attachment portion may be secured to one another.

The second pivot device and the mounting apparatus attachment portion may be attachable to one another by a connecting member. The second pivot device and the mounting apparatus attachment portion may be secured to one another by a connecting member. The connecting member may be a second connecting member.

The connecting member may be fastened to the second pivot device and the mounting apparatus attachment portion. The connecting member may be attachable to the second pivot device and the mounting apparatus attachment portion by one or more fastening devices. The connecting member may be secured to the second pivot device and the mounting apparatus attachment portion by one or more fastening devices. The one or more fastening devices may be mechanical fastening devices, or the like.

The connecting member may include a threaded portion. The connecting member may include a threaded portion at each end thereof. The connecting member may be fastened to the second pivot device and the mounting apparatus attachment portion by one or more nut members.

The one or more nut members being configured to engage with the threaded portion of the connecting member. The one or more nut members being configured to engage with the threaded portion of the connecting member and at least a portion of the second pivot device and the mounting apparatus attachment portion.

The connecting member may be an elongate member. The connecting member may be an elongate rod. The connecting member may be a rod member. The connecting member may be cylindrical elongate member. The connecting member may be a solid metal member.

The housing of the first arm portion may be a hollow member. The housing of the first arm portion may be a shell member.

The housing of the first arm portion may be a generally a cuboid-shaped member. The housing of the first arm portion may be a generally cuboid-shaped hollow member.

The housing of the first arm portion may be an extruded member.

The housing of the first arm portion may extend between the first pivot device and the second pivot device.

The housing of the first arm portion may be arranged to house the first biasing device and the second biasing device. The housing of the first arm portion may be arranged to house at least a portion of the first biasing device and at least a portion of the second biasing device.

The housing of the first arm portion may be configured to engage with the first pivot device. The housing of the first arm portion may be configured to engage with the first and second members of the first pivot device.

The housing of the first arm portion may be configured to engage and receive at least a portion of the first pivot device therein. The housing of the first arm portion may be configured to engage and receive at least a portion of the first and/or second member of the first pivot device therein. The housing of the first arm portion may be configured to engage and receive at least a portion of the second member of the first pivot device therein.

The housing of the first arm portion may be configured to engage with the second pivot device. The housing of the first arm portion may be configured to engage with the first and second members of the second pivot device.

The housing of the first arm portion may be configured to engage and receive at least a portion of the second pivot device therein. The housing of the first arm portion may be configured to engage and receive at least a portion of the first and/or second member of the second pivot device therein. The housing of the first arm portion may be configured to engage and receive at least a portion of the first member of the second pivot device therein.

The housing of the first arm portion may be configured to house the connecting member.

The housing of the first arm portion, the first and second pivot devices and the connecting member may be arranged such that housing may be engaged between the first and second pivot devices, and the connecting member may be secured/attached to the first and second pivot devices to apply a compressive force to the housing. That is, since the housing is located between the first and second pivot devices, as the connecting member is fastened to each pivot device, each pivot device moves slightly towards the other, which results in a compressive force being applied to each end of the housing. In this arrangement, the housing is clamped between the first and second pivot devices. The connecting member pulls the first and second pivot devices towards one another to clamp the components together.

The connecting member may be a tensioning member. The connecting member may be arranged to apply tension to the housing of the first arm portion and the first and second pivot devices. The connecting member may be arranged to apply compression to the housing of the first arm portion and the first and second pivot devices.

The housing of the first arm portion may include one or more apertures. The, or each, aperture may be an elongate aperture located on a side wall of the housing. The, or each, aperture may be an access aperture. That is, the, or each, aperture allows access to the inside of the housing.

The housing may include one or more inspection apertures. The, or each, inspection aperture may include a cover member. The cover member may be arranged to engage with the inspection aperture. The one or more inspection apertures may be located at a first, lower, end and/or a second, upper, end of the housing.

The housing of the first arm portion includes a recessed portion in a side wall thereof. The recessed portion may be a channel portion. The recessed portion may extend between a first, lower, end and a second, upper, end of the housing. The recessed portion may be a recessed channel that extends between a first, lower, end and a second, upper, end of the housing. The recessed portion may be arranged to be in the direction of the longitudinal axis of the first arm portion.

The recessed portion of the housing of the first arm portion may be arranged to receive one or more cables therein. The one or more cables may be operating cables of a medical device. The one or more cables may be power cables, communication cables, or the like. The one or more cables may be arranged to run between a first, lower, end and a second, upper, end of the housing. The one or more cables may be arranged to run in the direction of the longitudinal axis of the first arm portion.

The recessed portion of the housing of the first arm portion may include one or more cable clips. Each cable clip may be arranged to receive and secure at least a portion of a cable therein.

The housing of the second arm portion may be a hollow member. The housing of the second arm portion may be a shell member.

The housing of the second arm portion may be a generally a cuboid-shaped member. The housing of the second arm portion may be a generally cuboid-shaped hollow member. The housing of the second arm portion may be a generally cylindrical-shaped member. The housing of the second arm portion may be a generally cylindrical-shaped hollow member.

The housing of the second arm portion may be an extruded member.

The housing of the second arm portion may extend between the second pivot device and the mounting apparatus attachment portion.

The housing of the second arm portion may be configured to engage with the second pivot device. The housing of the second arm portion may be configured to engage with the first and second members of the second pivot device. The housing of the second arm portion may be configured to engage with the second member of the second pivot device.

The housing of the second arm portion may be configured to engage and receive at least a portion of the second pivot device therein. The housing of the second arm portion may be configured to engage and receive at least a portion of the first and/or second member of the second pivot device therein. The housing of the second arm portion may be configured to engage and receive at least a portion of the second member of the second pivot device therein.

The housing of the second arm portion may be configured to engage with the mounting apparatus attachment portion.

The housing of the second arm portion may be configured to engage and receive at least a portion of the mounting apparatus attachment portion therein.

The housing of the second arm portion may be configured to house the connecting member.

The housing of the second arm portion, the second pivot device, the mounting apparatus attachment portion and the connecting member may be arranged such that housing may be engaged between the second pivot device, the mounting apparatus attachment portion, and the connecting member may be secured/attached to the second pivot device and mounting apparatus attachment portion to apply a compressive force to the housing. That is, since the housing is located between the second pivot device and the mounting apparatus attachment portion, as the connecting member is fastened to the second pivot device and the mounting apparatus attachment portion, the second pivot device and the mounting apparatus attachment portion move slightly towards each other, which results in a compressive force being applied to each end of the housing. In this arrangement, the housing is clamped between the second pivot device and the mounting apparatus attachment portion. The connecting member pulls the second pivot device and the mounting apparatus attachment portion towards one another to clamp the components together.

The connecting member may be a tensioning member. The connecting member may be arranged to apply tension to the housing of the second arm portion, the second pivot device and the mounting apparatus attachment portion. The connecting member may be arranged to apply compression to the housing of the second arm portion, the second pivot device and the mounting apparatus attachment portion.

The housing may include one or more inspection apertures. The, or each, inspection aperture may include a cover member. The cover member may be arranged to engage with the inspection aperture. The one or more inspection apertures may be located at a first, lower, end and/or a second, upper, end of the housing.

The housing of the first arm portion includes a recessed portion in a side wall thereof. The recessed portion may be a channel portion. The recessed portion may extend between a first, lower, end and a second, upper, end of the housing. The recessed portion may be a recessed channel that extends between a first, lower, end and a second, upper, end of the housing. The recessed portion may be arranged to be in the direction of the longitudinal axis of the second arm portion.

The recessed portion of the housing of the second arm portion may be arranged to receive one or more cables therein. The one or more cables may be operating cables of a medical device. The one or more cables may be power cables, communication cables, or the like. The one or more cables may be arranged to run between a first, lower, end and a second, upper, end of the housing. The one or more cables may be arranged to run in the direction of the longitudinal axis of the second arm portion.

The recessed portion of the housing of the second arm portion may include one or more cable clips. Each cable clip may be arranged to receive and secure at least a portion of a cable therein.

The structure attachment portion may include a brake device. The brake device may be an electromagnetic brake device.

The brake device may be operable to hold the structure attachment portion in a plurality of different pivot positions relative to a base unit of a medical device system. The brake device may be operable prevent the structure attachment portion rotating relative to a base unit of a medical device system.

The brake device may be located between the structure attachment portion and a base unit of a medical device system.

The first pivot device may include a brake device. The brake device may be an electromagnetic brake device.

The brake device may be operable to hold the first and second members of the first pivot device in a plurality of different pivot positions relative to one another. The brake device may be operable to prevent the first and second members rotating relative to one another. The brake device may be operable to prevent the second member rotating relative to the first member.

The brake device may be located between the first and second members of the first pivot device.

The second pivot device may include a brake device. The brake device may be an electromagnetic brake device.

The brake device may be operable to hold the first and second members of the second pivot device in a plurality of different pivot positions relative to one another. The brake device may be operable to prevent the first and second members rotating relative to one another. The brake device may be operable to prevent the second member rotating relative to the first member.

The brake device may be located between the first and second members of the second pivot device.

The mounting apparatus attachment portion may include a brake device. The brake device may be an electromagnetic brake device.

The brake device may be operable to hold a mounting apparatus for a medical device in a plurality of different pivot positions relative to the mounting apparatus attachment portion. The brake device may be operable to prevent a mounting apparatus for a medical device rotating relative to the mounting apparatus attachment portion.

The brake device may be located between the mounting apparatus attachment portion and a mounting apparatus for a medical device.

The second arm portion may include a mounting apparatus attachment arm. The mounting apparatus attachment arm may be attachable to the mounting attachment portion of the second arm portion.

The mounting apparatus attachment arm may be attachable to a mounting apparatus. The mounting apparatus may be a mounting apparatus for a medical device.

The mounting apparatus attachment arm may be pivotably attachable to the mounting attachment portion of the second arm portion.

The mounting apparatus attachment portion may include a brake device. The brake device may be an electromagnetic brake device.

The brake device may be located between the mounting apparatus attachment portion and the mounting apparatus attachment arm.

The brake device may be operable to hold the mounting apparatus attachment arm in a plurality of different pivot positions relative to the mounting apparatus attachment portion. The brake device may be operable to prevent the mounting apparatus attachment arm rotating relative to the mounting apparatus attachment portion.

The mounting apparatus attachment arm may include a brake device. The brake device may be an electromagnetic brake device.

The brake device may be operable to hold a mounting apparatus for a medical device in a plurality of different pivot positions relative to the mounting apparatus attachment arm. The brake device may be operable to prevent a mounting apparatus for a medical device rotating relative to the mounting apparatus attachment arm.

The brake device may be located between the mounting apparatus attachment arm and a mounting apparatus for a medical device.

The first biasing device may be operable to pull the first arm portion from its second position to its first position. The first biasing device may be operable to exert a force on the first arm portion to move it from its second position to its first position.

The first biasing device may be one or more traction struts. The first biasing device may be one or more gas struts. The first biasing device may be one or more gas traction struts. The first biasing device may be one or more gas-operated struts. The first biasing device may be one or more gas springs.

The first biasing device may include one or more pistons and piston housings. The, or each, piston and piston housing may be gas-operated to exert a force on the piston to bias it towards the piston housing. The, or each, piston may move between a first position and a second position. The, or each, piston may be biased towards the first position. The first position may be an initial, or housed, position. The second position may be a final, or extended, position.

The first biasing device may be connected between the first and second pivot device. The first biasing device may be connected to the first and second pivot device. The first biasing device may be connected to the first member of the first pivot device and the first member of the second pivot device.

The, or each, piston housing of the first biasing device may be connected to the first member of the second pivot device and the, or each, piston of the first biasing device may be connected to the first member of the first pivot device. The piston housing may be connected by one or more fastening devices.

The first biasing device may include one or more cable members. The cable members may be wire cables, or the like. The one or more cable members may be pulley cables. The cable members may be metal cables, or the like.

The, or each, piston of the first biasing device may be connected to the first pivot device by a cable member. In this arrangement, the, or each, cable member is attached between the first pivot device and the, or each piston. The, or each, piston is indirectly connected to the first pivot device by a cable member. The, or each, piston is indirectly connected to the first member of the first pivot device by a cable member.

The cable member may be anchored to the, or each, piston of the first biasing device and the first member of the first pivot device. The cable member may be anchored by one or more fastening devices.

The, or each, cable member may be secured to a lower, or bottom, end of the first pivot device. The, or each, cable member may be secured to a lower, or bottom, end of the first member of the first pivot device.

The, or each, cable member may be arranged around an outwardly facing surface of the first pivot device. That is, the, or each, cable member may be located between a lower, or bottom, end of the first pivot device and an upper, or top, end of the first pivot device, and around an outer surface of the same between the two locations. The, or each, cable member may be arranged around an outwardly facing surface of the first member of the first pivot device. That is, the, or each, cable member may be located between a lower, or bottom, end of the first member of the first pivot device and an upper, or top, end of the first member of the first pivot device, and around an outer surface of the same between the two locations.

The first pivot device may include one or more pulley wheels. The pulley wheels may be configured to engage and support the, or each cable member. The second member of the first pivot device may include one or more pulley wheels. The pulley wheels may be configured to engage and support the, or each cable member. The one or more pulley wheels may be attached to the second member of the first pivot device.

The pulley wheels may be configured to engage and support the, or each cable member as the first arm portion moves between its first and second position.

The second biasing device may be operable to pull the second arm portion from its second position to its first position. The second biasing device may be operable to exert a force on the second arm portion to move it from its second position to its first position.

The second biasing device may be one or more traction struts. The second biasing device may be one or more gas struts. The second biasing device may be one or more gas traction struts. The second biasing device may be one or more gas-operated struts. The second biasing device may be one or more gas springs.

The second biasing device may include one or more pistons and piston housings. The, or each, piston and piston housing may be gas-operated to exert a force on the piston to bias it towards the piston housing. The, or each, piston may move between a first position and a second position. The, or each, piston may be biased towards the first position. The first position may be an initial, or housed, position. The second position may be a final, or extended, position.

The second biasing device may be connected between the first and second pivot device. The second biasing device may be connected to the first and second pivot device. The second biasing device may be connected to the second member of the first pivot device and the second member of the second pivot device.

The, or each, piston housing of the second biasing device may be connected to the second member of the first pivot device and the, or each, piston of the second biasing device may be connected to the second member of the second pivot device. The piston housing may be connected by one or more fastening devices.

The second biasing device may include one or more cable members. The cable members may be wire cables, or the like. The one or more cable members may be pulley cables. The cable members may be metal cables, or the like.

The, or each, piston of the second biasing device may be connected to the second pivot device by a cable member. In this arrangement, the, or each, cable member is attached between the second pivot device and the, or each piston. The, or each, piston is indirectly connected to the second pivot device by a cable member. The, or each, piston is indirectly connected to the second member of the second pivot device by a cable member.

The cable member may be anchored to the, or each, piston of the second biasing device and the second member of the second pivot device. The cable member may be anchored by one or more fastening devices.

The, or each, cable member may be secured to a lower, or bottom, end of the first pivot device. The, or each, cable member may be secured to an upper, or top, end of the second member of the second pivot device.

The, or each, cable member may be arranged around an outwardly facing surface of the second pivot device. That is, the, or each, cable member may be located between a lower, or bottom, end of the second pivot device and an upper, or top, end of the second pivot device, and around an outer surface of the same between the two locations. The, or each, cable member may be arranged to travel around an outwardly facing surface of the first member of the second pivot device. That is, the, or each, cable member may be located between a lower, or bottom, end of the first member of the second pivot device and an upper, or top, end of the first member of the second pivot device, and around an outer surface of the same between the two locations.

The second pivot device may include one or more pulley wheels. The pulley wheels may be configured to engage and support the, or each cable member. The first member of the second pivot device may include one or more pulley wheels. The pulley wheels may be configured to engage and support the, or each cable member. The one or more pulley wheels may be attached to the first member of the second pivot device.

The pulley wheels may be configured to engage and support the, or each cable member as the second arm portion moves between its first and second position.

The first pivot device may include a cover member. The cover member may be configured to cover the first and second members of the first pivot device.

The second pivot device may include a cover member. The cover member may be configured to cover the first and second members of the second pivot device.

The housing of the first arm portion may include a cover member. The cover member may be configured to cover the recessed portion of the housing.

The housing of the second arm portion may include a cover member. The cover member may be configured to cover the recessed portion of the housing.

The structure attachment portion may include a cover member.

The support structure may further comprise a mounting apparatus attachment arm. The mounting apparatus attachment arm may be attachable to the mounting apparatus attachment portion of the second arm portion. The mounting apparatus attachment arm may be configured to receive and engage a mounting apparatus for a medical device. The mounting apparatus attachment arm may be pivotably attachable to the mounting apparatus attachment portion. The mounting apparatus attachment arm be configured to pivotably engage with a mounting apparatus for a medical device.

The support structure may further comprise a mounting apparatus for a medical device. The mounting apparatus attachment arm may include a mounting apparatus for a medical device.

The support structure may further comprise a mounting apparatus for a medical device. The mounting apparatus may be attachable to the mounting apparatus attachment portion of the second arm portion.

The support structure may further comprise a medical device. The medical device may be attachable to the second arm portion of the support structure. The medical device may be attachable to the mounting apparatus attachment portion of the second arm portion. The medical device may be attachable to the mounting apparatus attachment arm of the second arm portion.

The medical device may be an X-ray tube. The medical device may be an X-ray tube head. The medical device may be a radiotherapy X-ray tube. The medical device may be a radiotherapy X-ray tube head. The medical device may be an end-window X-ray tube. The medical device may be an end-window X-ray tube head. The medical device may be an end-window radiotherapy X-ray tube. The medical device may be an end-window radiotherapy X-ray tube head.

The support structure may further comprise a base unit. The base unit may provide support to the support structure. The support structure may be attachable to the base unit.

The base unit may include one or more wheels, castors, or the like, to allow movement of the base unit.

The base unit may be a structure attachment portion. The structure attachment portion may be configured to be attached to a structure, such as a wall, wall member, or the like.

The support structure may further comprise a control system. The control system may be operable to control the operation of the support structure. The control system may be operable to control the operation of the first and second biasing devices, the brake devices and a medical device. The control system may be configured to provide power and/or data communication control signals to the components of the support structure.

The support structure may further comprise a control head. The control head may be a handle. The handle may be configured to allow the position of the first and second arm portions to be moved by a user. The control head may include one or more brake control switches. The brake control switches being operable to control the operation of the brake devices.

According to a second aspect of the present invention there is provided a support structure for a medical device system, comprising:
a first arm portion, the first arm portion including a structure attachment portion;
a second arm portion, the second arm portion being configured to support a medical device; and
a mounting apparatus for a medical device, the mounting apparatus being attachable to the second arm portion,
wherein the first arm portion is pivotably attachable to the structure attachment portion, and may pivot between a first position and a second position, and
wherein the second arm portion is pivotably attachable to the first arm portion, and may pivot between a first position and a second position, and
wherein the first arm portion includes:
   a first biasing device, the first biasing device being configured to bias the first arm portion towards its first position; and
   a second biasing device, the second biasing device being configured to bias the second arm portion towards its first position.

The support structure may further comprise a mounting apparatus attachment arm. The mounting apparatus attachment arm may be attachable to the second arm portion. The mounting apparatus attachment arm may be configured to receive and engage the mounting apparatus for a medical device. The mounting apparatus attachment arm may be pivotably attachable to the second arm portion. The mounting apparatus attachment arm be configured to pivotably engage with a mounting apparatus for a medical device.

The support structure may further comprise a medical device. The medical device may be attachable to the second arm portion of the support structure. The medical device may be attachable to the mounting apparatus of the second arm portion. The medical device may be attachable to the mounting apparatus attachment arm of the second arm portion.

The medical device may be an X-ray tube. The medical device may be an X-ray tube head. The medical device may be a radiotherapy X-ray tube. The medical device may be a radiotherapy X-ray tube head. The medical device may be an end-window X-ray tube. The medical device may be an end-window X-ray tube head. The medical device may be an end-window radiotherapy X-ray tube. The medical device may be an end-window radiotherapy X-ray tube head.

The support structure may further comprise a base unit. The base unit may provide support to the support structure. The support structure may be attachable to the base unit.

The base unit may include one or more wheels, castors, or the like, to allow movement of the base unit.

The base unit may be a structure attachment portion. The structure attachment portion may be configured to be attached to a structure, such as a wall, wall member, or the like.

The support structure may further comprise a control system. The control system may be operable to control the operation of the support structure. The control system may be operable to control the operation of the first and second biasing devices, the brake devices and a medical device. The control system may be configured to provide power and/or data communication control signals to the components of the support structure.

The support structure may further comprise a control head. The control head may be a handle. The handle may be configured to allow the position of the first and second arm portions to be moved by a user. The control head may include one or more brake control switches. The brake control switches being operable to control the operation of the brake devices.

Embodiments of the second aspect of the present invention may include one or more features of the first aspect of the present invention or its embodiments. Similarly, embodiments of the first aspect of the present invention may include one or more features of the second aspect of the present invention or its embodiments.

According to a third aspect of the present invention there is provided a medical device system, comprising:
a base unit; and
a support structure for a medical device system, comprising:
   a first arm portion, the first arm portion including a structure attachment portion; and
   a second arm portion, the second arm portion being configured to support a medical device,
   wherein the first arm portion is pivotably attachable to the structure attachment portion, and may pivot between a first position and a second position, and
   wherein the second arm portion is pivotably attachable to the first arm portion, and may pivot between a first position and a second position, and
   wherein the first arm portion includes:
      a first biasing device, the first biasing device being configured to bias the first arm portion towards its first position; and
      a second biasing device, the second biasing device being configured to bias the second arm portion towards its first position.

The base unit may provide support to the support structure. The support structure may be attachable to the base unit.

The base unit may include one or more wheels, castors, or the like, to allow movement of the base unit.

The base unit may be a structure attachment portion. The structure attachment portion may be configured to be attached to a structure, such as a wall, wall member, or the like.

The medical device system may further comprise a medical device. The medical device may be attachable to the second arm portion of the support structure. The medical device may be attachable to the mounting apparatus of the second arm portion. The medical device may be attachable to the mounting apparatus attachment arm of the second arm portion.

The medical device may be an X-ray tube. The medical device may be an X-ray tube head. The medical device may be a radiotherapy X-ray tube. The medical device may be a radiotherapy X-ray tube head. The medical device may be an end-window X-ray tube. The medical device may be an end-window X-ray tube head. The medical device may be an end-window radiotherapy X-ray tube. The medical device may be an end-window radiotherapy X-ray tube head.

The medical device system may further comprise a control system. The control system may be operable to control the operation of the support structure. The control system may be operable to control the operation of the first and second biasing devices, the brake devices and a medical device. The control system may be configured to provide power and/or data communication control signals to the components of the support structure.

The support structure may further comprise a control head. The control head may be a handle. The handle may be configured to allow the position of the first and second arm portions to be moved by a user. The control head may include one or more brake control switches. The brake control switches being operable to control the operation of the brake devices.

Embodiments of the third aspect of the present invention may include one or more features of the first or second aspects of the present invention or their embodiments. Similarly, embodiments of the first or second aspects of the present invention may include one or more features of the third aspect of the present invention or its embodiments.

According to a fourth aspect of the present invention there is provided a medical device system, comprising:
a base unit;
a support structure for a medical device system, comprising:
   a first arm portion, the first arm portion including a structure attachment portion; and
   a second arm portion, the second arm portion being configured to support a medical device,
   wherein the first arm portion is pivotably attachable to the structure attachment portion, and may pivot between a first position and a second position, and
   wherein the second arm portion is pivotably attachable to the first arm portion, and may pivot between a first position and a second position, and
   wherein the first arm portion includes:
      a first biasing device, the first biasing device being configured to bias the first arm portion towards its first position; and
      a second biasing device, the second biasing device being configured to bias the second arm portion towards its first position; and
a medical device, the medical device being attachable to the second arm portion of the support structure.

The base unit may provide support to the support structure. The support structure may be attachable to the base unit.

The base unit may include one or more wheels, castors, or the like, to allow movement of the base unit.

The base unit may be a structure attachment portion. The structure attachment portion may be configured to be attached to a structure, such as a wall, wall member, or the like.

The medical device may be attachable to the mounting apparatus of the second arm portion. The medical device may be attachable to the mounting apparatus attachment arm of the second arm portion.

The medical device may be an X-ray tube. The medical device may be an X-ray tube head. The medical device may be a radiotherapy X-ray tube. The medical device may be a radiotherapy X-ray tube head. The medical device may be an end-window X-ray tube. The medical device may be an end-window X-ray tube head. The medical device may be an end-window radiotherapy X-ray tube. The medical device may be an end-window radiotherapy X-ray tube head.

The medical device system may further comprise a control system. The control system may be operable to control the operation of the support structure. The control system may be operable to control the operation of the first and second biasing devices, the brake devices and a medical device. The control system may be configured to provide power and/or data communication control signals to the components of the support structure.

The support structure may further comprise a control head. The control head may be a handle. The handle may be configured to allow the position of the first and second arm portions to be moved by a user. The control head may include one or more brake control switches. The brake control switches being operable to control the operation of the brake devices.

Embodiments of the fourth aspect of the present invention may include one or more features of the first, second or third aspects of the present invention or their embodiments. Similarly, embodiments of the first, second or third aspects of the present invention may include one or more features of the fourth aspect of the present invention or its embodiments.

According to a fifth aspect of the present invention there is provided a support structure for a device system, comprising:
a first arm portion, the first arm portion including a structure attachment portion; and
a second arm portion, the second arm portion being configured to support a device,
wherein the first arm portion is pivotably attachable to the structure attachment portion, and may pivot between a first position and a second position, and
wherein the second arm portion is pivotably attachable to the first arm portion, and may pivot between a first position and a second position, and
wherein the first arm portion includes:
   a first biasing device, the first biasing device being configured to bias the first arm portion towards its first position; and
   a second biasing device, the second biasing device being configured to bias the second arm portion towards its first position.

The support structure may be for a medical device system.

Embodiments of the fifth aspect of the present invention may include one or more features of the first, second, third, or fourth aspects of the present invention or their embodiments. Similarly, embodiments of the first, second, third, or fourth aspects of the present invention may include one or more features of the fifth aspect of the present invention or its embodiments.

According to a sixth aspect of the present invention there is provided a method of operating a support structure for a medical device system, comprising the steps of:
providing a support structure for a medical device system, comprising:
   a first arm portion, the first arm portion including a structure attachment portion; and
   a second arm portion, the second arm portion being configured to support a medical device,
   wherein the first arm portion is pivotably attachable to the structure attachment portion, and may pivot between a first position and a second position, and
   wherein the second arm portion is pivotably attachable to the first arm portion, and may pivot between a first position and a second position, and
   wherein the first arm portion includes:
      a first biasing device, the first biasing device being configured to bias the first arm portion towards its first position; and
      a second biasing device, the second biasing device being configured to bias the second arm portion towards its first position;
operating the first biasing device to move the first arm portion between its first position and its second position; and
operating the second biasing device to move the second arm portion between its first position and its second position.

The support structure may further comprise a medical device. The medical device may be attachable to the second arm portion of the support structure. The medical device may be attachable to a mounting apparatus of the second arm portion. The medical device may be attachable to a mounting apparatus attachment arm of the second arm portion.

The medical device may be an X-ray tube. The medical device may be an X-ray tube head. The medical device may be a radiotherapy X-ray tube. The medical device may be a radiotherapy X-ray tube head. The medical device may be an end-window X-ray tube. The medical device may be an end-window X-ray tube head. The medical device may be an end-window radiotherapy X-ray tube. The medical device may be an end-window radiotherapy X-ray tube head.

Embodiments of the sixth aspect of the present invention may include one or more features of the first, second, third, fourth, or fifth aspects of the present invention or their embodiments. Similarly, embodiments of the first, second, third, fourth, or fifth aspects of the present invention may include one or more features of the sixth aspect of the present invention or its embodiments.

According to a seventh aspect of the present invention there is provided a device support system, comprising:
a base unit; and
a support structure for a device system, comprising:
   a first arm portion, the first arm portion including a structure attachment portion; and
   a second arm portion, the second arm portion being configured to support a device,
   wherein the first arm portion is pivotably attachable to the structure attachment portion, and may pivot between a first position and a second position, and
   wherein the second arm portion is pivotably attachable to the first arm portion, and may pivot between a first position and a second position, and
   wherein the first arm portion includes:
      a first biasing device, the first biasing device being configured to bias the first arm portion towards its first position; and
      a second biasing device, the second biasing device being configured to bias the second arm portion towards its first position.

Embodiments of the seventh aspect of the present invention may include one or more features of the first, second, third, fourth, fifth, or sixth aspects of the present invention or their embodiments. Similarly, embodiments of the first, second, third, fourth, fifth, or sixth aspects of the present invention may include one or more features of the seventh aspect of the present invention or its embodiments.

### BRIEF DESCRIPTION OF THE DRAWINGS

Embodiments of the invention will now be described, by way of example, with reference to the drawings, in which:

FIG. 1a is a perspective partial cut away side view of a support structure for a medical device in accordance with the present invention with a medical device mounted thereon;

FIG. 1b is an alternative perspective view of the support structure of FIG. 1a with the first and second arm portions in their first positions;

FIG. 1c is an alternative perspective view of the support structure of FIG. 1a with the first and second arm portions in their second;

FIG. 1d is a partial perspective side view of the support structure of FIG. 1a in a partially assembled state;

FIG. 1e is a further perspective side view of the support structure of FIG. 1a in a partially assembled state;

FIG. 1f is a further partial perspective side view of the support structure of FIG. 1a in a partially assembled state;

FIG. 1g is a further partial perspective side view of the support structure of FIG. 1a in a partially assembled state;

FIG. 1h is a further partial side view of the support structure of FIG. 1a in a partially assembled state;

FIG. 2a is a perspective side view of components of the first and second pivot devices of the support structure of FIG. 1a with connecting member;

FIG. 2b is a perspective side view of a component of the second pivot device of the support structure of FIG. 1a and the mounting apparatus attachment portion;

FIGS. 3a to 3c are side views of the first pivot device of the support structure of FIG. 1a in different states of operation;

FIGS. 4a to 4c are side views of the second pivot device of the support structure of FIG. 1a in different states of operation;

FIG. 5 is a perspective exploded view of the support structure of FIG. 1a; and

FIG. 6 is a schematic view of a medical device system according to the present invention.

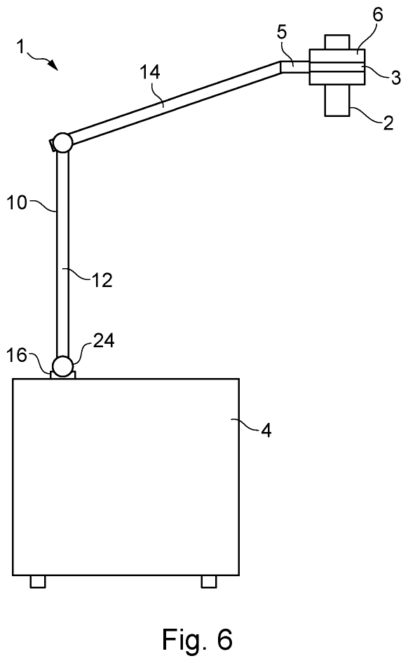

### DESCRIPTION OF PREFERRED EMBODIMENTS

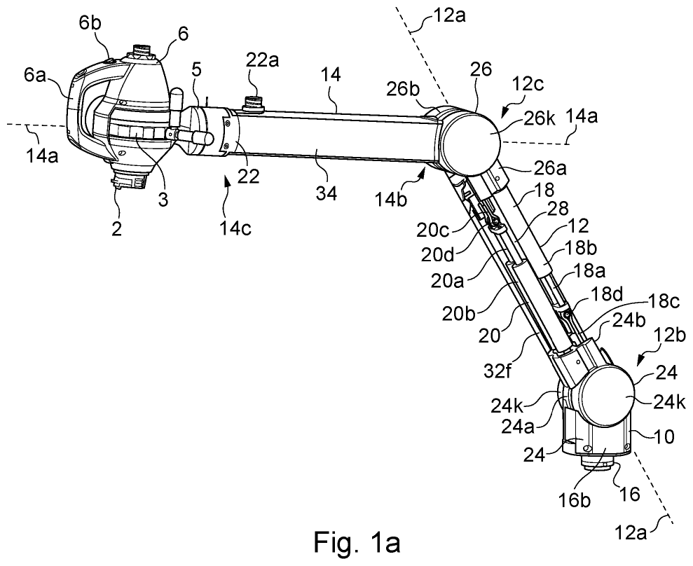

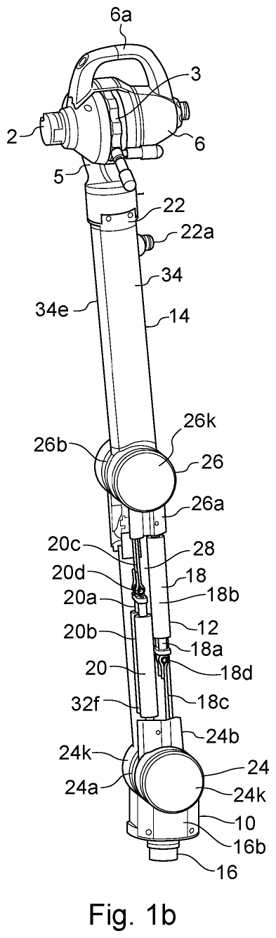

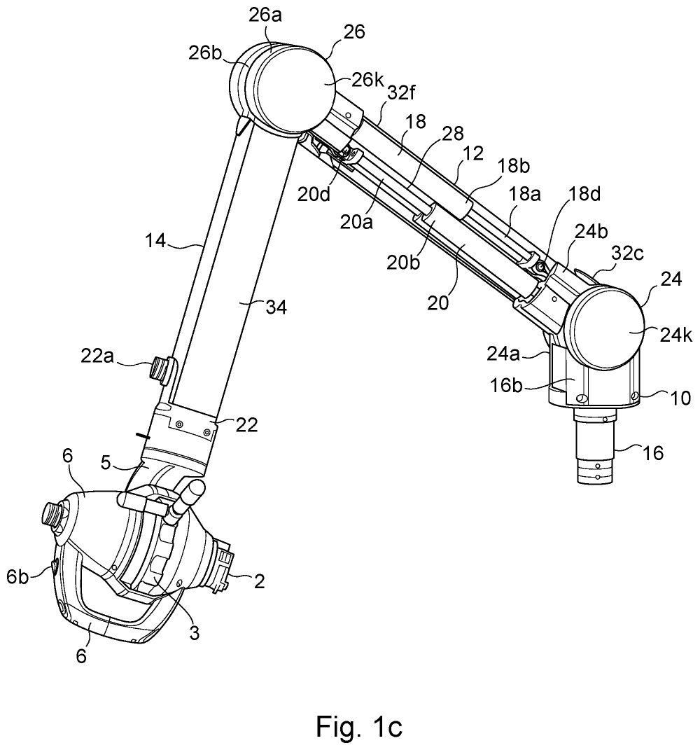

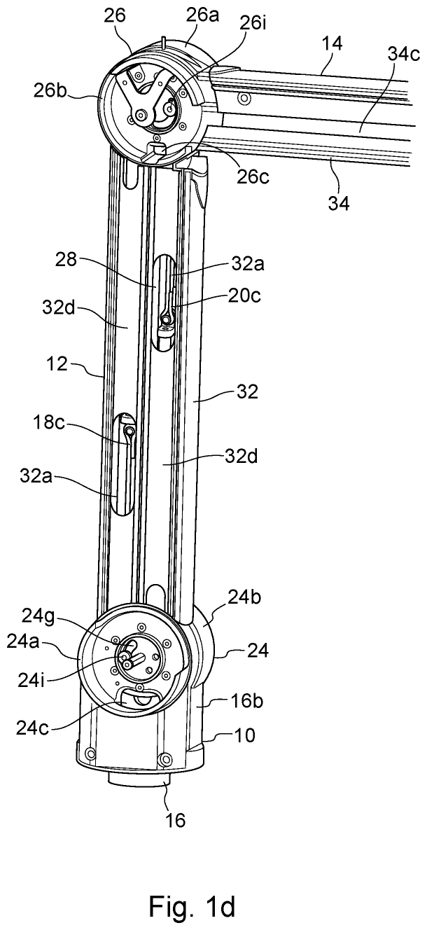

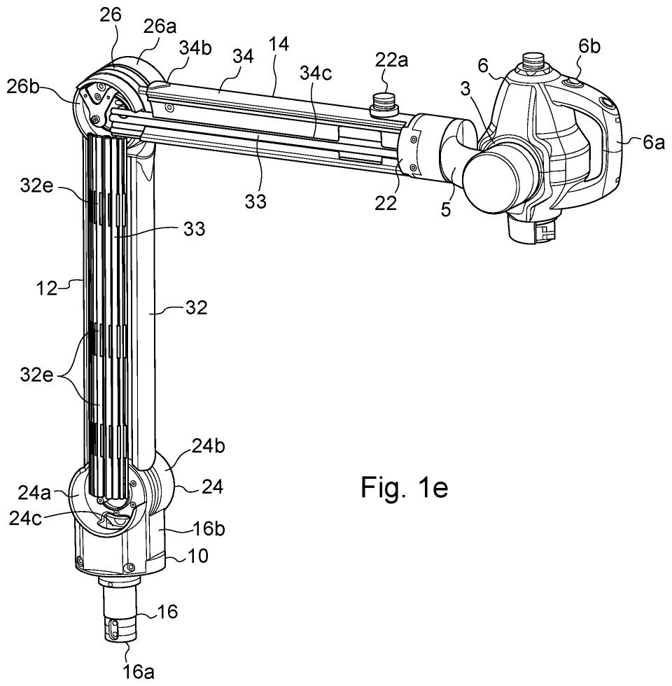

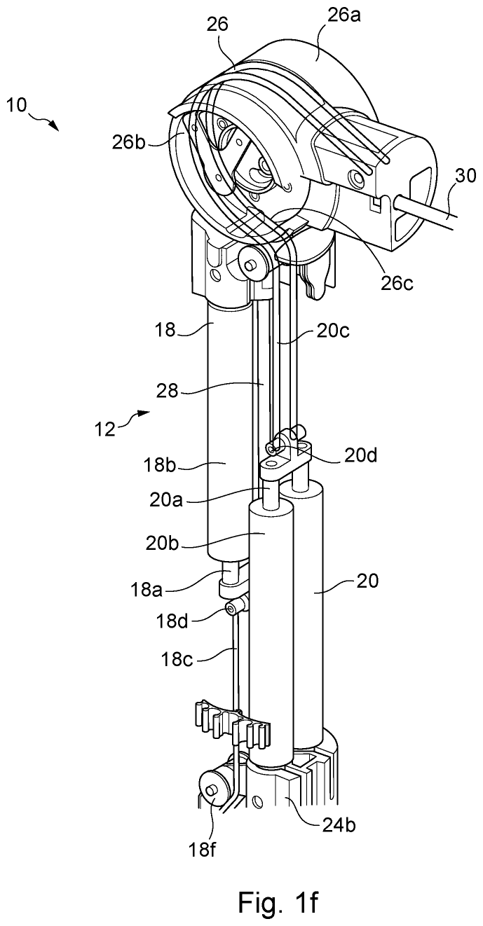

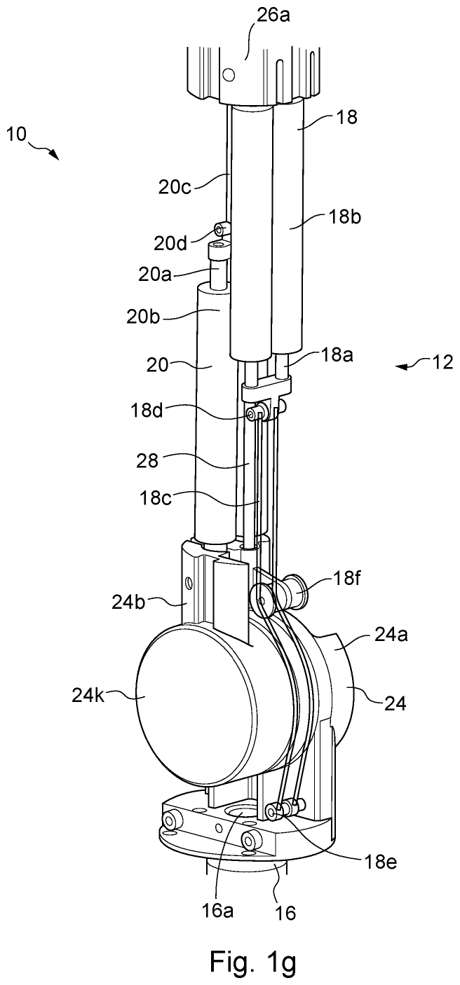

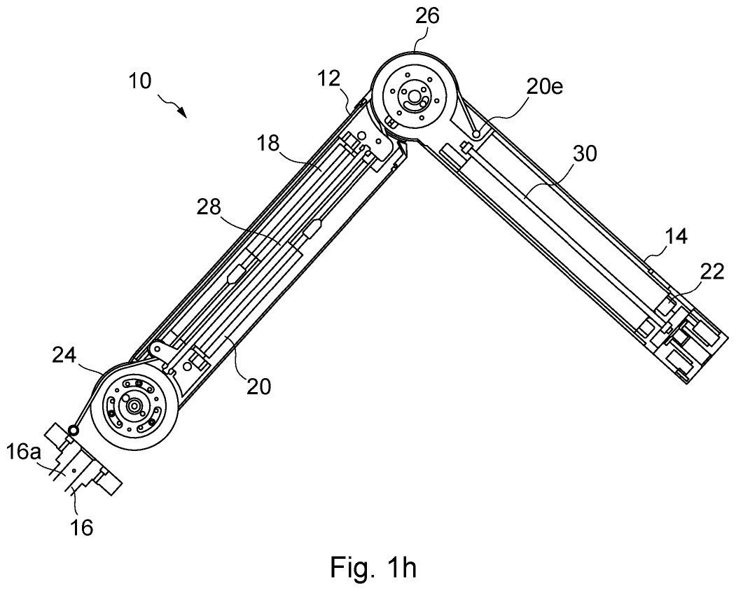

With reference to FIGS. 1a to 1 h, a support structure for a medical device system 10 is illustrated. The support structure 10 incudes a first arm portion 12 and a second arm portion 14.

The first arm portion 12 includes a structure attachment portion 16 and is pivotably attachable to the same. The first arm portion 12 may pivot between a first position (FIG. 1b) and a second position (FIG. 1c).

The second arm portion 14 is configured to support a medical device 2 and is pivotably attachable to the first arm portion 12. The second arm portion may pivot between a first position (FIG. 1b) and a second position (FIG. 1c).

The first arm portion 12 includes a first biasing device 18 and a second biasing device 20. The first biasing device 18 is configured to bias the first arm portion 12 towards its first position, and the second biasing device 20 is configured to bias the second arm portion 14 towards its first position.

As described further below, in the embodiment illustrated and described here, the support structure 10 is for an X-ray medical device system 1 (an example of a device system) and is configured to provide support to a medical device 2. The medical device 2 may be mounted on a mounting apparatus 3, which is supported by the support structure 10. The support structure 10 is a support arm and may be configurable to assume a plurality of different positions.

In the embodiment illustrated and described here, the medical device 2 is an X-ray tube head. The X-ray tube head may be a radiotherapy X-ray tube for the treatment of skin conditions, such as malignant or benign skin cancers or superficial skin lesions. However, it should be appreciated that the support structure 10 may be configured to provide support other devices, including other medical devices.

The first arm portion 12 is an elongate member and has a longitudinal axis 12a that extends between a first, lower, end 12b, and a second, upper, end 12c.

The second arm portion 14 is an elongate member and has a longitudinal axis 14a that extends between a first, lower, end 14b, and a second, upper, end 14c.

As described further below, the first and second arm portions 12, 14 are pivotably attachable to one another.

The structure attachment portion 16 is configured to allow the support structure 10 to be attachable to a structure. In the embodiment illustrated and described here, the structure is a base unit 4 of a medical device system 1. However, it should be appreciated that the structure may be a component of a building, such as a wall, or the like. Alternatively, the structure may be component of a vehicle, such as the wall of a building, such as a wall, or the like.

As illustrated, the structure attachment portion 16 is located at the lower end 12b of the first arm portion 12. The structure attachment portion 16 includes a through passage 16a. The through passage 16a allows cabling, or the like, to pass through the structure attachment portion 16.

The second support arm 14 is configured to provide support to the medical device 2. In the embodiment illustrated and described here, the second support arm 14 provides support to the medical device 2 through the mounting apparatus 3. The mounting apparatus 3 is configured to receive and engage with the medical device 2.

The second arm portion 14 includes a mounting apparatus attachment portion 22 located at the upper end 14c thereof. The mounting apparatus attachment portion 22 is configured to provide support to the mounting apparatus 3. The mounting apparatus 3 is attachable to the mounting apparatus attachment portion 22.

The mounting apparatus attachment portion 22 includes a through passage 22a. The through passage 22a allows cabling, or the like, to pass through the mounting apparatus attachment portion 22.

The first arm portion 12 is pivotably attachable to the structure attachment portion 16 by a first pivot device 24. The first pivot device 24 is located at the lower end 12b of the first arm portion 12 and is configured to allow the first arm portion 12 to pivot with respect to the structure attachment portion 16 between its first position (FIG. 1b) and its second position (FIG. 1c).

The first pivot device 24 includes a first pivot member 24a and a second pivot member 24b. The first pivot member 24a is attachable to the structure attachment portion 16. The second pivot member 24b is pivotably attachable to the first pivot member 24a. The second pivot member 24b is attachable to the first arm portion 12 at the lower end 12b thereof. In the embodiment illustrated and described here the first pivot member 24a is a pivot support member, or support member, and the second pivot member 24b is a pivot member. In this arrangement, the second pivot member 24b pivots relative to the first pivot member 24a.

The first pivot member 24a of the first pivot device 24 includes a through passage 24c. The through passage 24c allows cabling, or the like, to pass through the first pivot device 24.

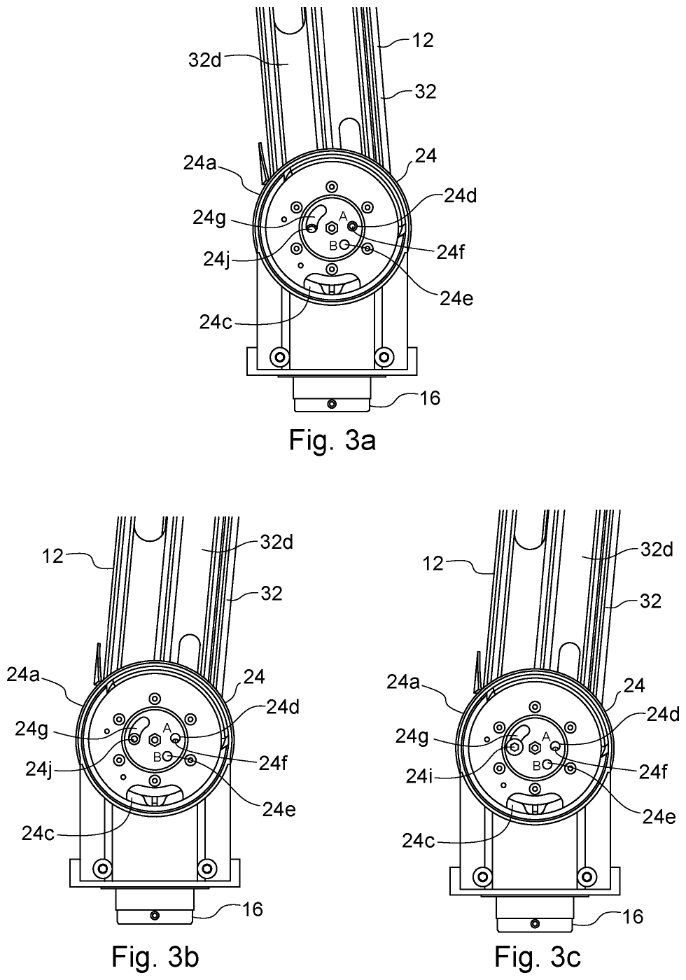

With reference to FIGS. 3a to 3c, the first pivot device 24 may be lockable, such that the first and second pivot members 24a, 24b cannot move relative to one another.

The first pivot member 24a includes a first locking aperture 24d and a second locking aperture 24e. The locking apertures 24d, 24e are angularly offset from one another and are arranged to allow a fastening member to pass therethrough. The second pivot member 24b includes a locking aperture 24f. The locking aperture 24f may be aligned with the first or second locking apertures 24d, 24e of the first pivot member 24a and is arranged to receive and engage with a fastening member. In this arrangement, the locking aperture 24f of the second pivot member 24b may operate with either the first locking aperture 24d or the second locking aperture 24e of the first pivot member 24a, such that the first pivot device 24 may be locked in two different positions, as described further below.

The first pivot member 24a of the first pivot device 24 includes a pivot aperture 24g, which is an arc-shaped slot that is arranged to allow a fastening member 24i therethrough.

The second pivot member 24b of the first pivot device 24 includes a pivot aperture 24j. The pivot aperture 24j is arranged to receive and engage with fastening member 24i. The fastening member 24i may be a bolt, or the like.

The first and second pivot members 24a, 24b of the first pivot device 24 may be arranged such that their respective pivot apertures 24g, 24j may be aligned, and the fastening member 24i may be inserted therein. In this arrangement the fastening member 24i is fixed to the second pivot member 24b of the first pivot device 24, and free to move relative to the first pivot member 24a of the first pivot device 24. The fastening member 24i is configured to abut either end of the pivot aperture 24g of the first pivot member 24a. A first abutment between the fastening member 24i and the first pivot member 24a defines the first position of the first pivot device 24, and a second abutment between the fastening member 24i and the first pivot member 24a defines the second position of the first pivot device 24. The first and second abutments are at opposite ends of the pivot aperture 24g of the first pivot member 24a. In this arrangement the length of the arc defines the range of pivot, or angular, motion of the second pivot member 24b relative to the first pivot member 24a of the first pivot device 24.

The first pivot device 24 may be arranged to allow the first arm portion 12 to pivot through between 30 and 70 degrees of rotation. The first pivot device 24 may be arranged to allow the first arm portion 12 to pivot through 50 degrees of rotation.

The second arm portion 14 is pivotably attachable to the first arm portion 12 by a second pivot device 26. The second pivot device 26 is located at the lower end 14b of the second arm portion 14 and is configured to allow the second arm portion 14 to pivot with respect to the first arm portion 12 between the first position (FIG. 1b) and the second position (FIG. 1c).

The second pivot device 26 includes a first pivot member 26a and a second pivot member 26b. The first pivot member 26a is attachable to the first arm portion 12. The second pivot member 26b is pivotably attachable to the first pivot member 26a. The second pivot member 26b is attachable to the second arm portion 14 at the lower end 14b thereof. In the embodiment illustrated and described here the first pivot member 26a is a pivot support member, or support member, and the second pivot member 26b is a pivot member. In this arrangement, the second pivot member 26b pivots relative to the first pivot member 26a.

The first pivot member 26a of the second pivot device 26 includes a through passage 26c. The through passage 26c allows cabling, or the like, to pass through the second pivot device 26.

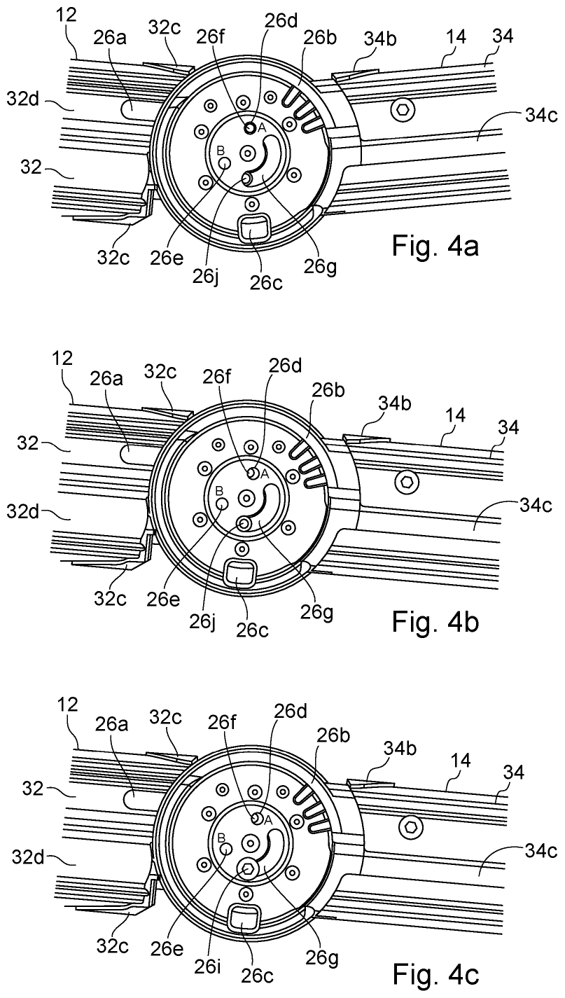

With reference to FIGS. 4a to 4c, the second pivot device 26 may be lockable, such that the first and second pivot members 26a, 26b cannot move relative to one another.

The second pivot member 26b includes a first locking aperture 26d and a second locking aperture 26e. The locking apertures 26d, 26e are angularly offset from one another and are arranged to allow a fastening member to pass therethrough. The first pivot member 26a includes a locking aperture 26f. The locking aperture 26f may be aligned with the first or second locking apertures 26d, 26e of the second pivot member 26b and is arranged to receive and engage with a fastening member. In this arrangement, the locking aperture 26f of the first pivot member 26a may operate with either the first locking aperture 26d or the second locking aperture 26e of the second pivot member 26b, such that the second pivot device 26 may be locked in two different positions, as described further below.

The second pivot member 26b of the second pivot device 26 includes a pivot aperture 26g, which is an arc-shaped slot that is arranged to allow a fastening member 26i therethrough.

The first pivot member 26a of the second pivot device 26 includes a pivot aperture 26j. The pivot aperture 26j is arranged to receive and engage with fastening member 26i. The fastening member 26i may be a bolt, or the like.

The first and second pivot members 26a, 26b of the second pivot device 26 may be arranged such that their respective pivot apertures 26g, 26j may be aligned, and the fastening member 26i may be inserted therein. In this arrangement the fastening member 26i is fixed to the first pivot member 26a of the second pivot device 26, and free to move relative to the second pivot member 26b of the second pivot device 26. The fastening member 26i is configured to abut either end of the pivot aperture 26g of the second pivot member 26b. A first abutment between the fastening member 26i and the second pivot member 26b defines the first position of the second pivot device 26, and a second abutment between the fastening member 26i and the second pivot member 26b defines the second position of the second pivot device 26. The first and second abutments are at opposite ends of the pivot aperture 26g of the second pivot member 26b. In this arrangement the length of the arc defines the range of pivot, or angular, motion of the second pivot member 26b relative to the first pivot member 26a of the second pivot device 26.

The second pivot device 26 may be arranged to allow the second arm portion 14 to pivot through between 100 and 120 degrees of rotation. The second pivot device 26 may be arranged to allow the second arm portion 14 to pivot through 110 degrees of rotation.

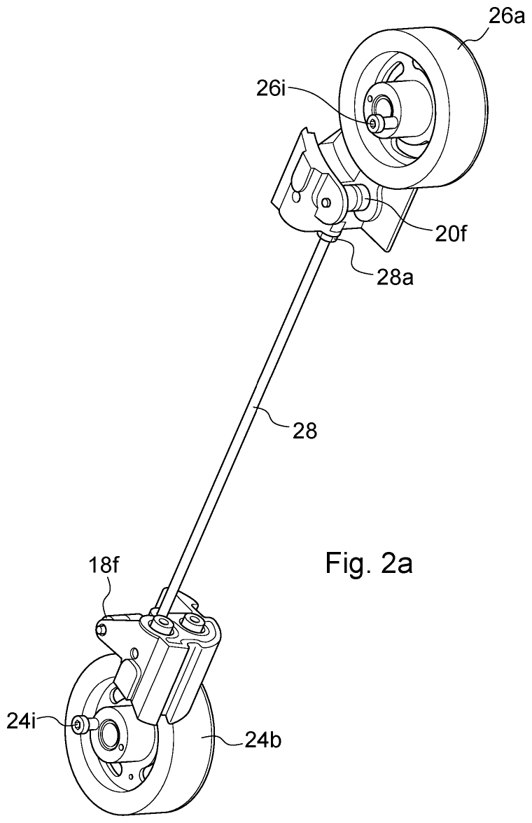

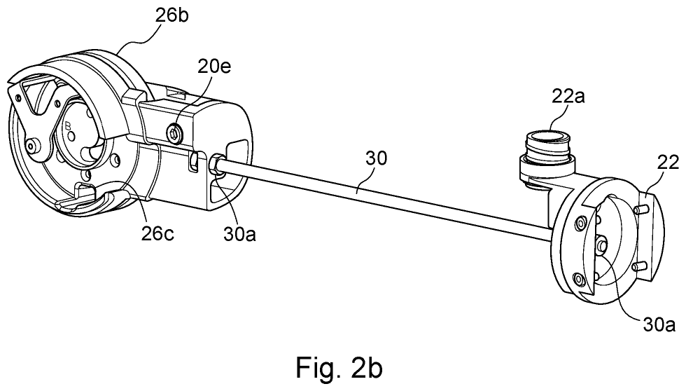

As best illustrated in FIGS. 2a and 2b, the first and second pivot devices 24, 26 are attachable to one another by a connecting member 28. The connecting member 28 is attachable to each of the first and second pivot devices 24, 26 by one or more fastening devices 28a. In the embodiment illustrated and described here the connecting member 28 includes a threaded portion at each end, and the connecting member 28 is fastened to the first and second pivot devices 24, 26 by one or more nut members. The one or more nut members being configured to engage with the threaded portion of the connecting member 28 and at least a portion of each pivot device 24, 26. In this arrangement a force can be applied to each pivot device 24, 26 when the nut members are tightened on the thread of the connecting member 28.

In the embodiment illustrated and described here, the connecting member 28 is a metal rod. However, it should be appreciated that other suitable connecting members may be used.

As best illustrated in FIGS. 2a and 2b, the second pivot device 26 and the mounting apparatus attachment portion 22 are attachable to one another by a connecting member 30. The connecting member 30 is attachable to the second pivot device 26 and the mounting apparatus attachment portion 22 by one or more fastening devices 30a. In the embodiment illustrated and described here the connecting member 30 includes a threaded portion at each end, and the connecting member 30 is fastened to the second pivot device 26 and the mounting apparatus attachment portion 22 by one or more nut members. The one or more nut members being configured to engage with the threaded portion of the connecting member 30 and at least a portion of the second pivot device 26 and the mounting apparatus attachment portion 22. In this arrangement a force can be applied to the second pivot device 26 and the mounting apparatus attachment portion 22 when the nut members are tightened on the thread of the connecting member 30.

In the embodiment illustrated and described here, the connecting member 30 is a metal rod. However, it should be appreciated that other suitable connecting members may be used.

The first arm portion 12 includes a housing 32. The housing 32 is a generally cuboid-shaped hollow member, and, as described further below, houses the first and second biasing devices 18, 20 and the connecting member 28.

The housing 32 extends between the first pivot device 24 and the second pivot device 26. In the embodiment illustrated and described here, the housing 32 is configured to engage and receive at least a portion of the second pivot member 24b of the first pivot device 24 therein, and at least a portion of the first pivot member 26a of the second pivot device 26 therein. The housing 32 engages with both the first pivot device 24 and the second pivot device 26.

The housing 32, the first and second pivot devices 24, 26 and the connecting member 28 are arranged such that the connecting member 28 may be secured/attached to the first and second pivot devices 24, 26 to apply a compressive force to the housing 32. That is, since the housing 32 is located between the first and second pivot devices 24, 26, as the connecting member 28 is fastened to each pivot device 24, 26, each pivot device 24, 26 moves slightly towards the other, which results in a compressive force being applied to each end of the housing 32. In this arrangement, the housing 32 is clamped between the first and second pivot devices 24, 26. The connecting member 28 pulls the first and second pivot devices 24, 26 towards one another to clamp the components together. The connecting member 28 may be a tensioning member, which applies a compressive force to the housing 32 of the first arm portion 12 and the first and second pivot devices 24, 26.

The housing 32 includes apertures 32a. The apertures 32a, are elongate apertures and are located on a side wall of the housing 32. The apertures 32a allow access to the inside of the housing for maintenance, and the like.

The housing 32 includes inspection apertures 32b. The inspection apertures 32b allow the inside of the housing 32 to be viewed. The inspection apertures 32b include a cover member 32c. The inspection apertures 32b are located at lower and upper ends of the housing 32.

The housing 32 includes a recessed portion 32d in a side wall thereof. The recessed portion 32d is a channel portion that extends between a lower end and an upper end of the housing 32. The recessed portion 32d is arranged in the direction of the longitudinal axis 12a of the first arm portion 12.

As best illustrated in FIG. 1e, the recessed portion 32d of the housing 32 is arranged to receive one or more cables 33 therein. The cables 33 may be operating cables of the medical device 2, power cables, communication cables, and the like. Cable clips 32e are provided to secure the cables 33 to the recessed portion 32d.

The second arm portion 14 includes a housing 34. The housing 34 is a generally cuboid-shaped hollow member which houses the connecting member 30.

The housing 34 extends between the second pivot device 26 and the mounting apparatus attachment portion 22. In the embodiment illustrated and described here, the housing 34 is configured to engage and receive at least a portion of the second pivot member 26b of the second pivot device 26 therein, and at least a portion of the mounting apparatus attachment portion 22. The housing 34 engages with both the second pivot device 26 and the mounting apparatus attachment portion 22.

The housing 34, the second pivot device 26, the mounting apparatus attachment portion 22 and the connecting member 30 are arranged such that the connecting member 30 may be secured/attached to the second pivot device 26 and mounting apparatus attachment portion 22 to apply a compressive force to the housing 34. That is, since the housing 34 is located between the second pivot device 26 and the mounting apparatus attachment portion 22, as the connecting member 30 is fastened to the second pivot device 26 and the mounting apparatus attachment portion 22, the second pivot device 26 and the mounting apparatus attachment portion 22 move slightly towards each other, which results in a compressive force being applied to each end of the housing 34. In this arrangement, the housing 34 is clamped between the second pivot device 26 and the mounting apparatus attachment portion 22. The connecting member 30 pulls the second pivot device 26 and the mounting apparatus attachment portion 22 towards one another to clamp the components together. The connecting member 30 may be a tensioning member, which applies a compressive force to the housing 34 of the second arm portion 14 and the second pivot device 26 and the mounting apparatus attachment portion 22.

The housing 34 includes inspection apertures 34a. The inspection apertures 34a allow the inside of the housing 34 to be viewed. The inspection apertures 34a include a cover member 34b. The inspection apertures 34a are located at lower and upper ends of the housing 34.

The housing 34 includes a recessed portion 34c in a side wall thereof. The recessed portion 34c is a channel portion that extends between a lower end and an upper end of the housing 34. The recessed portion 34c is arranged in the direction of the longitudinal axis 14a of the second arm portion 14.

As best illustrated in FIG. 1e, the recessed portion 34c of the housing 34 is arranged to receive one or more cables 33 therein. The cables 33 may be operating cables of the medical device 2, power cables, communication cables, and the like. Cable clips 34d are provided to secure the cables 33 to the recessed portion 34c.

It will be appreciated that the cables 33 run from the medical device 2, through the mounting apparatus attachment portion 22, along the recessed portion 34c of the housing 34, through the second pivot member 26b of the second pivot device 26, along the recessed portion 32d of the housing 32, through the first pivot member 24a of the first pivot device 24 and through the structure attachment portion 16.

The structure attachment portion 16 may include a brake device (not illustrated), the brake device being operable to hold the structure attachment portion 16 in a plurality of different pivot positions, and prevent movement thereof, relative to the base unit 4 of the medical device system 1. The brake device may be an electromagnetic brake device. The brake device is located between the structure attachment portion 16 and the base unit 4 of the medical device system 1.

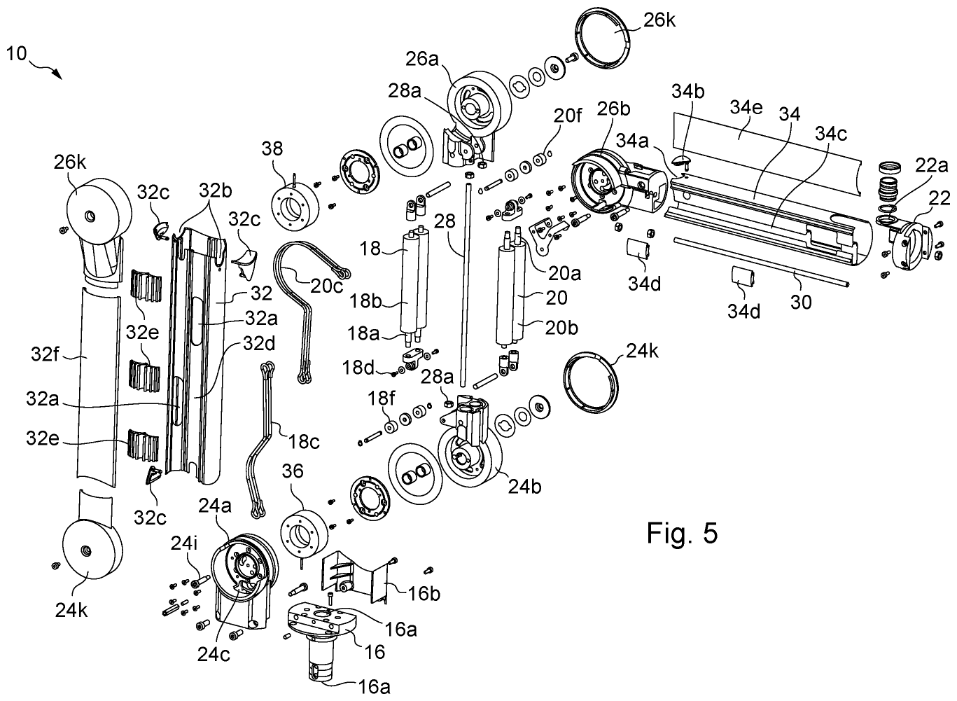

With reference to FIG. 5, the first pivot device 24 includes a brake device 36, the brake device 36 being operable to hold the first and second pivot members 24a, 24b of the first pivot device 24 in a plurality of different pivot positions, relative to one another. The brake device 36 may be an electromagnetic brake device. The brake device 36 may be located between the first and second pivot members 24a, 24b of the first pivot device 24.

With reference to FIG. 4, the second pivot device 26 includes a brake device 38, the brake device 38 being operable to hold the first and second pivot members 26a, 26b of the second pivot device 26 in a plurality of different pivot positions, relative to one another. The brake device 38 may be an electromagnetic brake device. The brake device 38 may be located between the first and second pivot members 26a, 26b of the first pivot device 26.

The mounting apparatus attachment portion 22 may include a brake device (not shown), the brake device being operable to hold the mounting apparatus 3 in a plurality of different pivot positions relative to the mounting apparatus attachment portion 22. The brake device may be an electromagnetic brake device. The brake device may be located between the mounting apparatus attachment portion 22 and a mounting apparatus 3.

The second arm portion 24 may include a mounting apparatus attachment arm 5, the mounting apparatus attachment arm 5 may be attachable to the mounting apparatus attachment portion 22 of the second arm portion 14. The mounting apparatus attachment arm 5 may also be attachable to a mounting apparatus 3. The mounting apparatus attachment arm 5 may be pivotably attachable to the mounting apparatus attachment portion 22 of the second arm portion 14. The mounting apparatus attachment portion 22 may include a brake device (not shown) between it and the mounting apparatus attachment arm 5. The brake device may be an electromagnetic brake device. The brake device may be operable to hold the mounting apparatus attachment arm 5 in a plurality of different pivot positions relative to the mounting apparatus attachment portion 22. The brake device may be operable to prevent the mounting apparatus attachment arm 5 rotating relative to the mounting apparatus attachment portion 22.

With reference to FIGS. 1a to 1h, and as described further below, the first biasing device 18 is operable to exert a force on the first arm portion 12 to move it from its second position to its first position, and the second biasing device 20 is operable to exert a force on the second arm portion 14 to move it from its second position to its first position.

In the embodiment illustrated and described here, the first biasing device 18 includes two traction struts. The traction struts are gas-operated.

However, if should be appreciated that other suitable types of biasing devices may be used.

Each traction strut 18 includes a piston 18a and a piston housing 18b. The piston 18a is configured to be biased towards the piston housing 18b. The housed position of the piston 18a in the piston housing 18b may be an initial, or first, position, and the exposed position of the piston 18a out of the piston housing 18b may be an extended, or second, position.

The first biasing device 18 is connected between the first and second pivot devices 24, 26. The piston housings 18b are connected to first pivot member 26a of the second pivot device 26, and the pistons 18a are connected to the first pivot member 24a of the first pivot device 24 by cable members 18c.

As best illustrated in FIGS. 1f to 1g, the cable members 18c are attached to the pistons 18a by fastening devices 18d and attached to the first pivot member 24a by a fastening device 18e (see FIG. 1g). The fastening devices 18d, 18e are bolts that anchor the cable members 18c to the pistons 18a and pivot member 24a.

The cable members 18c are metal wire cables. However, it should be appreciated that other suitable types of cables may be used.

As illustrated, the cable members 18c extend from the pistons 18a, over pulley wheels 18f mounted on the second pivot member 24b of the first pivot device 24, around an outer surface of the pivot members 24a, 24b of the first pivot device 24, before being anchored to the first pivot member 24a. The pulley wheels 18f engage and support the cable members 18c as the first arm portion 12 moves between its first and second position.

Again, in the embodiment illustrated and described here, the second biasing device 20 includes two traction struts. The traction struts are gas-operated. However, if should be appreciated that other suitable types of biasing devices may be used.

Again, each traction strut 20 includes a piston 20a and a piston housing 20b. The piston 20a is configured to be biased towards the piston housing 20b. The housed position of the piston 20a in the piston housing 20b may be an initial, or first, position, and the exposed position of the piston 20a out of the piston housing 20b may be an extended, or second, position.

The second biasing device 20 is connected between the first and second pivot devices 24, 26. The piston housings 20b are connected to the second pivot member 24b of the first pivot device 24, and the pistons 20a are connected to the second pivot member 26b of the second pivot device 26 by cable members 20c.

As best illustrated in FIGS. 1f to 1g, the cable members 20c are attached to the pistons 20a by fastening devices 20d and attached to the second pivot member 26b by a fastening device 20e (see FIG. 1h and 2b). The fastening devices 20d, 20e are bolts that anchor the cable members 20c to the pistons 20a and pivot member 26b.

The cable members 20c are metal wire cables. However, it should be appreciated that other suitable types of cables may be used.

As illustrated, the cable members 20c extend from the pistons 20a, over pulley wheels 20f mounted on the first pivot member 26b of the second pivot device 26, around an outer surface of the pivot members 26a, 26b of the second pivot device 26, before being anchored to the second pivot member 26b. The pulley wheels 20f engage and support the cable members 20c as the second arm portion 14 moves between its first and second position.

The first pivot device 24 includes a cover member 24k. The cover member 24k is configured to cover the first and second pivot members 24a, 24b of the first pivot device 24.

The second pivot device 26 includes a cover member 26k. The cover member 26k is configured to the first and second pivot members 26a, 26b of the second pivot device 26.

The housing 32 of the first arm portion 12 includes a cover member 32f. The cover member 32f is configured to cover the recessed portion 32d of the housing 32.

The housing 34 of the second arm portion 14 includes a cover member 34e. The cover member 34e is configured to cover the recessed portion 34c of the housing 34.

The structure attachment portion 16 may include a cover member 16b.

The support structure 10 may further include a control system (not shown). The control system may be operable to control the operation of the support structure 10. The control system may be operable to control the operation of the brake devices 36, 38 and the medical device 2. The control system may be configured to provide power and/or data communication control signals to the components of the support structure 10. The control system may be located in the base unit 4 of the X-ray system 1.

The support structure 10 may further comprise a control head 6. The control head 6 be a housing member with a handle portion 6a. The handle 6a may be configured to allow the position of the first and second arm portions 12, 14 to be moved by a user. The control head 6 may include one or more brake control switches 6b. The brake control switches 6b being operable to control the operation of the brake devices 36, 38. In use, the brake control switches 6b are operated to release the brake devices 36, 38 so that the position of the first and second arm portions 12, 14 may be adjusted, and the medical device 2 may be positioned as required by the user.

With reference to FIGS. 1b and 1c, the operation of the support structure 10 will now be described.

In FIG. 1b, the support structure 10 is illustrated in an initial position. In this position the first and second arm portions 12, 14 are arranged substantially vertically and colinearly. In this position the first and second biasing devices 18, 20 are in their first position. That is, the pistons 18a, 20a of each traction strut 18, 20 are substantially entirely located in their piston housings 18b, 20b. In this position, the traction struts 18, 20 may hold the first and second arms 12, 14 in this position. In this position the brake devices 36, 38 may also be operational to brake the first and second pivot devices 24, 26.

In order to move the first and second arm portions 12, 14 and medical device 2 to a desired position, a user grasps the handle 6a of the control head 6 and then operates the brake control switches 6b to release the brake devices 36, 38. With the brake devices 36, 38 released the user can move the control head 6 and medical device 5 to the desired location.

FIG. 1c illustrates a repositioning of the support structure 10 where the first arm portion 12 has been moved from its first position to its second position, and the second arm portion 14 has been moved from its first position to its second position. As illustrated, in this position the first and second biasing devices 18, 20 are in their first and second position. That is, the pistons 18a, 20a of each traction strut 18, 20 are extended from their piston housings 18b, 20b. In this position, the traction struts 18, 20 may hold the first and second arms 12, 14 in this position. In this position the brake devices 36, 38 may also be operational to brake the first and second pivot devices 24, 26.

As described above, the first pivot device 24 is arranged to allow the first arm portion 12 to pivot through between 30 and 70 degrees of rotation. The second pivot device 26 is arranged to allow the second arm portion 14 to pivot through between 100 and 120 degrees of rotation.

With the control head 6 and medical device 5 in the desired position, the user can operate the medical device 5 as required.

With reference to FIGS. 3a to 3c and 4a to 4c, the first and second arm portions 12, 14 may be arranged in a number of different positions, depending on the state of use of the support structure 10.

FIG. 3a illustrates the position where the first arm portion 12 is pivoted with respect to the first pivot device 24, such that the locking aperture 24f of the second pivot member 24b is aligned with the first locking aperture 24d (A) of the first pivot member 24a. In this position, the first arm portion 12 may be locked in position by inserting a fastening member into apertures 24d and 24f. The fastening member locks the first and second pivot members 24a, 24b together. This position is termed an assembly position of the support structure 10 and the first arm portion 12 is under no tension or bias. This allows the various components of the support structure to be assembled. In this position, if the biasing device 18 is fitted, it is in its first position and no force is applied to the first arm portion 12.

FIG. 3b illustrates the position where the first arm portion 12 is pivoted with respect to the first pivot device 24, such that the pivot aperture 24g of the first pivot member 24a is aligned with the pivot aperture 24j of the second pivot member 24b. In this position, the biasing device 18 is moved slightly from its first position to create tension in the cables 18c. With the biasing device 18 held in this position, the fastening member 24i is fastened to the pivot aperture 24g, as illustrated in FIG. 3c. This position may be termed the operating position. When the first arm portion 12 is in its second position, and the fastening member 24i abuts the end portion of the pivot aperture 24g, the second locking aperture 24e (B) of the first pivot member 24a may be aligned with the locking aperture 24f of the second pivot member 24b. In this position, the first arm portion 12 may be locked in position by inserting a fastening member into 24e and 24f. The fastening member locks the first and second pivot members 24a, 24b together. This position is termed transit, or transport, position of the support structure 10.

FIG. 4a illustrates the position where the second arm portion 14 is pivoted with respect to the second pivot device 26, such that the locking aperture 26f of the first pivot member 24a is aligned with the first locking aperture 26d (A) of the second pivot member 26b. In this position, the second arm portion 14 may be locked in position by inserting a fastening member into apertures 26d and 26f. The fastening member locks the first and second pivot members 26a, 26b together. This position is termed an assembly position of the support structure 10 and the second arm portion 14 is under no tension or bias. This allows the various components of the support structure to be assembled. In this position, if the biasing device 20 is fitted, it is in its first position and no force is applied to the second arm portion 14.

FIG. 4b illustrates the position where the second arm portion 14 is pivoted with respect to the second pivot device 26, such that the pivot aperture 26g of the second pivot member 26b is aligned with the pivot aperture 26j of the first pivot member 26a. In this position, the biasing device 20 is moved slightly from its first position to create tension in the cables 20c. With the biasing device 20 held in this position, the fastening member 26i is fastened to the pivot aperture 26j, as illustrated in FIG. 4c. This position may be termed the operating position. When the second arm portion 14 is in its second position, and the fastening member 26i abuts the end portion of the pivot aperture 26g, the second locking aperture 26e (B) of the second pivot member 26b may be aligned with the locking aperture 26f of the first pivot member 26a. In this position, the second arm portion 14 may be locked in position by inserting a fastening member into 26e and 26f. The fastening member locks the first and second pivot members 26a, 26b together. This position is termed transit, or transport, position of the support structure 10.

Providing a support structure 10 for a medical device system as described above reduces the size and weight of the support arms. Providing first and second biasing devices 18, 20 in the first support arm 12 means that the second support arm 14 can be reduced in size and weight. This means that the arm has a reduced moment of inertia, which means that is easier to move the support arm and hold the arm in a balanced, equilibrium position. Reducing the weight of the support arm also means that the size of the componentry required to operate the arm may be reduced. This means that lower cost componentry may be used. Also, smaller componentry means that smaller housings can be used, which reduces the cost of the same.

Furthermore, providing connecting members 28, 30 between the first and second pivot devices 24, 26 improves the torsional rigidity of the first and second support arms 12, 14. The connecting members 28, 30 also improves the transmission of load through the structure 10.

Also, providing housing members 32, 34 with recessed portions 32d, 34c for cables 33 allows easy access to the cables 33 for assembly and maintenance. In this arrangement, the cables 33 are captive within the support structure 10, which prevents unauthorised access thereto. There is also no interference with the function of the support structure range of movement by the cables 33.

Furthermore, providing access apertures 32a, 34a in housings 32, 34 allows easy access to cables 18c, 20c for assembly and maintenance.

Also, providing inspection apertures 32b, 34a in housings 32, 34 allows easy inspection of the componentry for monitoring, maintenance and assembly purposes.

Modifications and improvements may be made to the above without departing from the scope of the present invention.

For example, although the structure attachment portion 16 has been illustrated and described above as being attachable to a structure which is a base unit 4 of a medical device system, it should be appreciated that the structure may be a component of a building, such as a wall, or the like. Alternatively, the structure may be component of a vehicle, such as the wall of a building, such as a wall, or the like. In this arrangement, the support structure 10 may be attachable to a wall, vehicle wall, or the like.

Furthermore, although the support structure 10 has been illustrated and describe above as being used to support a medical device 2, it should be appreciated that the support structure 10 may be configured to provide support to other types of devices, medical or otherwise.

Also, although the first and second biasing devices 18, 20 have been illustrated and described above as being gas-operated traction struts, it should be appreciated that other suitable types of springs, struts or biasing devices may be used.

1. A support structure for a medical device system, comprising:
a first arm portion, the first arm portion including a structure attachment portion; and
a second arm portion, the second arm portion being configured to support a medical device,
wherein the first arm portion is pivotably attachable to the structure attachment portion, and may pivot between a first position and a second position, and
wherein the second arm portion is pivotably attachable to the first arm portion, and may pivot between a first position and a second position, and
wherein the first arm portion includes:
a first biasing device, the first biasing device being configured to bias the first arm portion towards its first position; and
a second biasing device, the second biasing device being configured to bias the second arm portion towards its first position.

2. (canceled)

3. The support structure of claim 1, wherein the first arm portion is pivotably attachable to the structure attachment portion by a first pivot device, the first pivot device being configured to allow the first arm portion to pivot with respect to the structure attachment portion between the first position and the second position,
wherein the first pivot device includes a first pivot member and a second pivot member, the first pivot member being attachable to the structure attachment portion, the second pivot member being pivotably attachable to the first pivot member, and the second pivot member being attachable to the first arm portion,
wherein the second arm portion is pivotably attachable to the first arm portion by a second pivot device, the second pivot device being configured to allow the second arm portion to pivot with respect to the first arm portion between the first position and the second position,
wherein the second pivot device includes a first pivot member and a second pivot member, the first pivot member being attachable to the first arm portion, the second pivot member being pivotably attachable to the first pivot member, and the second pivot member being attachable to the second arm portion.

4-6. (canceled)

7. The support structure of claim 1, wherein the second arm portion is pivotably attachable to the first arm portion by a second pivot device, the second pivot device being configured to allow the second arm portion to pivot with respect to the first arm portion between the first position and the second position,
wherein the second pivot device includes a first pivot member and a second pivot member, the first pivot member being attachable to the first arm portion, the second pivot member being pivotably attachable to the first pivot member, and the second pivot member being attachable to the second arm portion.

8. (canceled)

9. The support structure of claim 7, wherein the second pivot device is lockable, such that the first and second pivot members cannot pivot relative to one another.

10. The support structure of claim 9, wherein the second pivot device is lockable in more than one position, such that the first and second pivot members of the second pivot device are lockable in a number of different positions relative to one another.

11. The support structure of claim 3,
wherein the first and second pivot devices are attachable to one another by a connecting member, the connecting member being fastened to each of the first and second pivot devices, and
wherein the second pivot member of the first pivot device is attachable to the first pivot member of the second pivot device by the connecting member.

12. (canceled)

13. The support structure of claim 7, wherein the second arm portion includes a mounting apparatus attachment portion, the mounting apparatus attachment portion being configured to support a medical device,
wherein the second pivot device and the mounting apparatus attachment portion are attachable to one another by a connecting member, the connecting member being fastened to the second pivot device and the mounting apparatus attachment portion,
wherein the second pivot member of the second pivot device is attachable to the mounting apparatus attachment portion by the connecting member.

14. (canceled)

15. The support structure of claim 11, wherein the first arm portion includes a housing, the housing being arranged to extend between the first pivot device and the second pivot device and to house the first and second biasing devices,
wherein the housing of the first arm portion, the first and second pivot devices and the connecting member are arranged such that housing is engaged between the first and second pivot devices, and the connecting member is secured to the first and second pivot devices to apply a compressive force to the housing.

16-18. (canceled)

19. The support structure of claim 13, wherein the second arm portion includes a housing, the housing being arranged to extend between the second pivot device and mounting apparatus attachment portion,
wherein the housing of the second arm portion, the second pivot device, the mounting apparatus attachment portion and the connecting member are arranged such that housing is engaged between the second pivot device, the mounting apparatus attachment portion, and the connecting member is secured to the second pivot device and mounting apparatus attachment portion to apply a compressive force to the housing.

20-22. (canceled)

23. The support structure of claim 3, wherein the first biasing device is connected to the first pivot member of the first pivot device and the first pivot member of the second pivot device.

24. The support structure of claim 3, wherein the first biasing device includes one or more pistons and piston housings, the, or each, piston being biased towards its housing,
wherein the, or each, piston housing of the first biasing device is connected to the first pivot member of the second pivot device and the, or each, piston of the first biasing device is connected to the first pivot member of the first pivot device.

25. (canceled)

26. The support structure of claim 24, wherein the first biasing device includes one or more cable members, the, or each, piston of the first biasing device being connected to the first pivot device by the cable member.

27. The support structure of claim 26, wherein the, or each, cable member is arranged around an outwardly facing surface of the first pivot member of the first pivot device, such that the, or each, cable member is located between a lower end of the first pivot member of the first pivot device and an upper end of the first pivot member of the first pivot device, and around an outer surface of the same between the two locations.

28. The support structure of claim 26, wherein the second member of the first pivot device includes one or more pulley wheels, the pulley wheels being the pulley wheels being configured to engage and support the, or each, cable member.

29. The support structure of claim 3, wherein second biasing device is connected to the second pivot member of the first pivot device and the second pivot member of the second pivot device.

30. The support structure of any of claim 3, wherein the second biasing device includes one or more pistons and piston housings, the, or each, piston being biased towards its housing,
wherein the, or each, piston housing of the second biasing device is connected to the second pivot member of the first pivot device and the, or each, piston of the second biasing device is connected to the second pivot member of the second pivot device.

31. (canceled)

32. The support structure of claim 30, wherein the second biasing device includes one or more cable members, the, or each, piston of the second biasing device being connected to the second pivot device by the cable member.

33. The support structure of claim 32, wherein the, or each, cable member is arranged around an outwardly facing surface of the second pivot member of the second pivot device, such that the, or each, cable member is located between a lower end of the first pivot member of the second pivot device and an upper end of the first pivot member of the second pivot device, and around an outer surface of the same between the two locations.

34. The support structure of claim 32, wherein the first pivot member of the second pivot device includes one or more pulley wheels, the pulley wheels being the pulley wheels being configured to engage and support the, or each, cable member.

35. A medical device system, comprising:
a base unit;
a support structure for a medical device system, comprising:
a first arm portion, the first arm portion including a structure attachment portion; and
a second arm portion, the second arm portion being configured to support a medical device,
wherein the first arm portion is pivotably attachable to the structure attachment portion, and may pivot between a first position and a second position, and
wherein the second arm portion is pivotably attachable to the first arm portion, and may pivot between a first position and a second position, and
wherein the first arm portion includes:
a first biasing device, the first biasing device being configured to bias the first arm portion towards its first position; and
a second biasing device, the second biasing device being configured to bias the second arm portion towards its first position; and
a medical device, the medical device being attachable to the second arm portion of the support structure.

36. (canceled)
